(19) United States
(12) Patent Application Publication
(10) Pub. No.: US 2011/0027200 A1
(43) Pub. Date: Feb. 3, 2011

(54) METHODS TO STABILIZE AND PREVENT BREAKDOWN OF SUNSCREEN AND OTHER TOPICAL AND ORAL PREPARATIONS AND COMPOSITIONS PRODUCED THEREBY

(76) Inventor: Eric F. Bernstein, Gladwyne, PA (US)

Correspondence Address: Kathleen A. Tyrrell;Licata & Tyrrell P.C., 66 E. Main Street, Marlton, NJ, 08053, US

(21) Appl. No.: 12/847,177

(22) Filed: Jul. 30, 2010

Related U.S. Application Data
(60) Provisional application No. 61/229,935, filed on Jul. 30, 2009.

(51) Int. Cl. A61K 8/49; A61K 38/06; A61K 31/341; A61K 36/82; A61K 31/355; A61K 31/07; A61K 31/01; A61K 36/87; A61K 31/385; A61K 31/194; A61K 31/715; A61K 36/48; A61K 31/045; A61K 33/24; A61K 31/445; A61P 17/18; A61P 31/00; A61Q 17/04

(52) U.S. Cl. 424/59; 514/21.9; 514/474; 424/729; 514/458; 514/725; 514/762; 424/766; 514/440; 514/574; 514/53; 424/757; 514/729; 424/617; 514/315

(57) ABSTRACT

Methods for stabilizing, maintaining the activity of and inhibiting growth of microorganisms in sunscreen preparations, anti-oxidant preparations and topical and oral pills and supplements by addition of at least one molecule chosen from nitroxides and nitrones and compositions produced thereby are provided.

This patent application claims the benefit of priority from U.S. Provisional Application Ser. No. 61/229,935, filed Jul. 30, 2009, teachings of which are herein incorporated by reference in their entirety.

### FIELD OF THE INVENTION

The present disclosure is related to preventing breakdown, stabilizing and preserving sunscreen compounds and/or preventing free-radical scavenging compounds from breakdown.

### BACKGROUND OF THE INVENTION

Sunscreens are typically composed of various compounds that absorb or reflect light to protect the skin from the deleterious effects of solar radiation, most importantly ultraviolet radiation. Typical sunscreen ingredients include but are not limited to: avobenzone (Parsol 1789), dioxybenzone, ecamsule, methyl anthranilate, meradimate, oxybenzone, benzophenone, benzophenone-3, sulisobenzone, titanium dioxide, zinc oxide, faminobenzoic acid, cinnamates, octyl methoxycinnamate (OMC), ethylhexyl p-Methoxycinnamaet, cinoxate, dioxybenzone, ensulizole, homosalate, octocrylene, octinoxate, octisalate, oxybenzone, octyl dimethyl paba, padimate 0, para-aminobenzoic acid (PABA), salicylates, octyl salicylate (OCS), sulisobenzone, and torlamine salycilate.

Breakdown of sunscreen ingredients dramatically reduces their effectiveness over time in the sun. As more ingredients are added to sunscreen preparations, interaction of the ingredients with each other as well as breakdown of certain sunscreen ingredients due to sun-exposure is an ongoing problem. Breakdown of sunscreen ingredients and a resulting loss of sun protection or a decrease in the sun protection factor (SPF) results from direct damage to sunscreen by them absorbing ultraviolet radiation or from free radicals generated within the sunscreen causing breakdown of sunscreen ingredients and a decrease in SPF. Breakdown of sunscreens can also occur from other environmental exposure of sunscreens to free-radicals or from aging of sunscreen products.

Accordingly, there is a need for better free-radical quenchers and sun absorbers in sunscreen products. Such a quencher would help solve the problem of breakdown or loss of effectiveness of sunscreen ingredients. Nitroxides or nitrones could reduce the potential degradation or inactivation of sunscreen ingredients from free radical exposure, ultraviolet radiation exposure, interaction of sunscreen ingredients, aging of the product, or interaction of any ingredients in the sunscreen formulation.

### SUMMARY OF THE INVENTION

To reduce the breakdown or inactivation of sunscreens or sunscreen components, topical preparations, oral medications, and/or supplement preparations, the present invention is directed to stabilization thereof using nitroxides and/or nitrones.

### DETAILED DESCRIPTION OF THE INVENTION

From their early chemistry until the present time, nitrones have been largely employed in Organic Chemistry. The importance of the nitrone functionality has been revealed by the growing number of scientific papers which appeared in the literature over the last years concerning nitrones and related compounds. Most of the work based on the nitrone functional group is related to the field of the Organic Synthesis (both concerning cycloaddition reactions and nucleophilic additions). A comprehensive review of nitrone synthesis is provided in Science of Synthesis, 2004, Vol. 27. p. 511, which is herein incorporated by reference.

As a general topic, the chemistry of nitrones has been described in the past, including concerning cycloaddition chemistry, and a more detailed description of the nitrone functionality in various volumes in the Patai series. The Chemistry of Carbon-Nitrogen Double Bond Patai, S. (Ed.), John Wiley & sons, New York, 1969. (b) The Chemistry of double bonded functional groups, Patai, S. (Ed.), John Wiley & sons, New York, 1977, Supplement A. (c) Breuer, E. Nitrones and nitronic acid derivatives: an update In Nitrones, Nitronates and Nitroxides, Patai, S.; Rappoport, Z. (Eds.), John Wiley & sons, New York, 1989, Supplement U2, Chapter 3, pp. 245-312, all of which are herein incorporated by reference.

Nitrones are easily prepared by several methods well-documented in the primary literature, such as in Franco, S.; Merchan, F. L.; Merino, P. Tejero, T. Synth. Commun. 1995, 25, 2275-2284 (b) Dondoni, A.; Franco, S.; Junquera, F.; Merchan, F. L.; Merino, P. Tejero, T. Synth. Commun. 1994, 25, 2537-2550. This reference, which is herein incorporated by reference, describes the often used condensation reactions between carbonyl compounds and oxidation of amines, imines or hydroxylamines. See, also, Murahashi, S. I. Angew. Chem. Int. Ed. Engl. 1995, 34, 2443-2465. (b) Murahashi, S. I.; Mitsui, H.; Shiota, T.; Tsuda, T.; Watanabe, S. J. Org. Chem. 1990, 55, 1736-1744, which are herein incorporated by reference

Nitroxide and nitrone compounds have been shown to have significant free-radical scavenging abilities. These agents have been shown to prevent oxidative damage in a number of systems and have been used to preserve a number of materials including paints and rubber. Many nitroxides and nitrone compounds have anti-oxidant properties, and function as triplet quenchers in dye lasers.

The inventor herein has now found that nitroxides or nitrones such as, but not limited to Tempol [4-hydroxy-2,2,6,6-tetramethyl-piperidinyloxy, free radical] can be used in a sunscreen to stabilize the sunscreen formulation thereby reducing the breakdown of sunscreen agents, and reducing the decrease in effectiveness of sunscreens as a function of sun exposure, free-radical exposure, aging of the sunscreen, or interaction of sunscreen components. Other non-limiting examples of compounds that can be used according to the present disclosure include water soluble, anti-oxidants, such as glutathione and its derivatives, and n-acetyl compounds, such as n-acetyl cysteine.

In addition, in accordance with the present invention, nitroxides or nitrones may be used to augment the activity of other sunscreens or preserve their ability to function as desired by combining them with agents including, but not limited to, avobenzone (Parsol 1789), dioxybenzone, ecamsule, methyl anthranilate, meradimate, oxybenzone, benzophenone, benzophenone-3, sulisobenzone, titanium dioxide, zinc oxide, faminobenzoic acid, cinnamates, octyl methoxycinnamate (OMC), ethylhexyl p-Methoxycinnamaet, cinoxate, dioxybenzone, ensulizole, homosalate, octocrylene, octinoxate, octisalate, oxybenzone, octyl dimethyl paba, padimate O, para-aminobenzoic acid (PABA), salicylates, octyl salicylate (OCS), sulisobenzone, and torlamine salycilate or other sunscreen ingredients.

Accordingly, there is disclosed in the present invention a means for preserving the function of sunscreen ingredients or anti-oxidants, said means comprising addition of a nitroxide or nitrone compound.

In one embodiment, the nitroxide comprises tempol [4-hydroxy-2,2,6,6-tetramethyl-piperidinyloxy, free radical].

In another embodiment, the nitroxide or nitrone compound is selected to be combined with agents including, but not limited to, avobenzone (Parsol 1789), dioxybenzone, ecamsule, methyl anthranilate, meradimate, oxybenzone, benzophenone, benzophenone-3, sulisobenzone, titanium dioxide, zinc oxide, faminobenzoic acid, cinnamates, octyl methoxycinnamate (OMC), ethylhexyl p-Methoxycinnamaet, cinoxate, dioxybenzone, ensulizole, homosalate, octocrylene, octinoxate, octisalate, oxybenzone, octyl dimethyl paba, padimate O, para-aminobenzoic acid (PABA), salicylates, octyl salicylate (OCS), sulisobenzone, and torlamine salycilate and their derivates but not limited to them.

The disclosed compounds can be used to generally improve the performance and life span of a formulation to which it is added.

U.S. Pat. No. 5,840,734 describes the use of Tempol in prevention of photoaging, sunburn and skin cancer caused by the UVA and UVB rays of sunlight. Additionally, U.S. Pat. No. 6,552,040 describes using other nitroxides in addition to Tempol for photoprotection, and describes using nitroxides including Tempol to augment wound healing. Also U.S. Pat. Nos. 7,314,633, 7,285,544, 7,229,629, 6,552,040, and 6,338,855 describe the use of nitroxides in skin disease.

In the method of the present invention, a nitroxide, such as tempol [4-hydroxy-2,2,6,6-tetramethyl-piperidinyloxy, free radical] or a nitrone is added to a sunscreen preparation for stabilization. In one embodiment, the nitroxide or nitrone is added in an amount from about 3% to about 10% of the total formulation. In one embodiment, the nitroxide or nitrone is added to a sunscreen preparation containing avobenzone and/or octinoxate.

In other embodiments, there is disclosed a method of improving the performance or life span of topical products containing anti-oxidants by adding the described nitroxide or nitrone to the formulation of the particular topical skin preparation including but not limited to lotions, gels, solutions, creams, ointments, emulsions, and sprays. For example, when used in topical product, the method may comprise adding at least one nitroxide or nitrone to the topical product to retain anti-oxidant activity of the product for a longer period of time by protecting the other ingredients in the product from free-radical or ultraviolet radiation damage.

In other embodiments, there is disclosed a method of improving the performance or life span of any topical products by adding the described nitroxide or nitrone to the formulation of the particular topical skin preparation including but not limited to lotions, gels, solutions, creams, ointments, emulsions, and sprays. For example, when used in topical product may comprise adding at least one nitroxide or nitrone to the topical product so that it retains the original properties of the product for a longer period of time and protecting the other ingredients in the product from free-radical or ultraviolet radiation damage.

In other embodiments, there is disclosed a method of improving the life span of any topical products by adding the described nitroxide or nitrone to the formulation of the particular topical skin preparation including but not limited to lotions, gels, solutions, creams, ointments, emulsions, and sprays for the purpose of limiting growth of microorganisms and limiting the ability of micro-organisms to metabolize product ingredients. Microorganisms include but are not limited to bacteria, prions, yeast, fungi and viruses. For example, when used in topical product may comprise adding at least one nitroxide or nitrone to topical product to prevent growth of micro-organisms or metabolism of product constituents by micro-organisms.

Addition of a nitroxide and nitrone to an oral medication or supplement preparation is also expected to stabilize or maintain activity of or prevent growth of microorganisms in an oral medication or supplement preparation. Examples of oral medication or supplement include, but are not limited to, pills, powders, capsules, dissolvable pills or capsules, liquids, and gels.

Various in vitro and in vivo models can be used to demonstrate the ability of nitrones or nitroxides to prevent breakdown, stabilize and preserve sunscreen compounds and/or prevent free-radical scavenging compounds from breakdown and/or to increase longevity and/or maintain activity and/or prevent growth of microorganism in topical preparations and oral preparations.

For example, a nitroxide such as Tempol can be added to a suncreen preparation. This preparation is then subjected to conditions such as, but not limited to, radiation known to breakdown and decrease efficacy of the sunscreen preparation. This preparation can then be tested in an in vitro or in vivo model for sunscreen agents. Such models include, but are in no way limited to, cells models of photodamage and the mouse edema ear model. Preferred is that this nitroxide or nitrone containing preparation be compared to a sunscreen preparation without the nitroxide or nitrone exposed to the same conditions known to breakdown and decrease efficacy of the sunscreen preparation to which the nitroxide or nitrone is added just before application to the model. Further testing can be performed in humans in accordance with established procedures.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention.

The following nonlimiting example is provided to further illustrate the present invention.

### Example

### Use of Tempol as a Stabilizing Additive to Sunscreen Formulations

These experiments were performed in accordance with procedures described by Stanfield, et al. in Photochem. Photobiol. Sci., 2010, 9, 489-494.

The photostability standard contained the following:

3% Avobenzone and 4% Octinoxate in 95% ethanol.

For these experiments, 1 MED=2 SEDs (20 effective mJ/cm², or 2.39 unweighted J/cm²); Irradiance 13.3 mw/cm²; Exposure time for 1 MED: 179 seconds. UV exposures were administered using a 150 watt solar simulator with a liquid light guide (16 mm beam).

Approximately 20 mg of standard was applied to a 5 cm×5 cm roughened PMMA plate and absorbance was computed using a reference plate with 8 mg of petrolatum.

Applied and transmitted Spectral irradiance values were measured with an OL756 spectroradiometer with a 6 inch integrating sphere, calibrated for a 16 mm source.

Baseline data for this photostability standard were first determined. Specifically, the change in optical characteristics when the applied formulation was irradiated using multiples of MED ranging from 0 to 13, where 1 MED is an amount of simulated solar ultraviolet radiation that would produce a minimal erythema in previously unexposed “Type 1 or 2” human skin, was determined.

Tempol was then added to solutions of 3% avobenzone and 4% octinoxate in ethanol at concentrations ranging from 0.00316% to 10%. While addition of Tempol reduced the concentrations of avobenzone and octinoxate thus lowering the absorbance, sunscreen preparations with 10% or 3% added Tempol retained more of their protective value (SPF) than the preparation without Tempol. See Table 1.

TABLE 1

| | Solution # | 0 | 10 | 11 |
|---|---|---|---|---|
| | % Tempol | 0.0000% | 10.0000% | 3.1600% |
| Net SPF After Irradiation | SPF | 10.36 | 5.78 | 5.92 |
| with 1 MED | | | | |
| Before Irradiation | SPF0 | 14.11 | 7.06 | 7.69 |
| | % Change | −26.58% | −18.15% | −22.99% |

Other embodiments of the invention will be apparent to those skilled in the art from consideration of the specification. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method for stabilizing or increasing longevity of or maintaining activity of sunscreen ingredients in a sunscreen preparation, said method comprising adding to the preparation at least one molecule chosen from nitroxides and nitrones.

2. The method of claim 1, wherein said nitroxides and nitrones are mixed with other sunscreen ingredients in a sunscreen formulation to stabilize the sunscreen ingredients.

3. The method of claim 1, where in the sunscreen preparation is a lotion, gel, solution, cream, ointment, emulsion, or spray.

4. The method of claim 1, wherein the nitroxide comprises tempol [4-hydroxy-2,2,6,6-tetramethyl-piperidinyloxy, free radical].

5. The method of claim 4 wherein the tempol is added at an amount of 3% to 10% of the total formulation.

6. The method of claim 1 wherein the sunscreen preparation comprises avobenzone and/or octinoxate.

7. A sunscreen preparation with enhanced stability or increased activity or longevity produced in accordance with the method of claim 1.

8. A method for stabilizing or increasing longevity of or maintaining activity of a topical preparation containing free-radical scavengers or anti-oxidants, said method comprising adding to the topical preparation at least one molecule chosen from nitroxides and nitrones.

9. The method of claim 8, wherein the anti-oxidants are selected from the group of glutathione, n-acetyl compounds, ascorbic acid, green tea, alpha-tocophorel, retinol, lycopene, serine protease inhibitors, alpha-1-antitrypsin, anti-chymotrypsin, grape seed extract, alpha-lipoic acid, citric acid, lactobionic acid, licorice, beta carotene, polyphenols, lutein, zeaxanthin, selenium, and derivatives thereof.

10. The method of claim 8, where in the topical preparation is a lotion, gel, solution, cream, ointment, emulsion, or spray.

11. The method of claim 8, wherein the nitroxide comprises tempol [4-hydroxy-2,2,6,6-tetramethyl-piperidinyloxy, free radical].

12. A topical preparation with enhanced stability or increased activity or longevity produced in accordance with the method of claim 8.

13. A method for stabilizing or increasing the longevity of or maintaining activity of topical preparations, oral medications or supplement preparations, said compound comprising adding to the topical preparation, oral medication or supplement preparation at least one molecule chosen from nitroxides and nitrones.

14. The method of claim 13, where in the topical preparation is a lotion, gel, solution, cream, ointment, emulsion, or spray.

15. The method of claim 13 where in the oral medication or supplement is a pill, powder, capsule, dissolvable pill or capsule, liquid, or gel.

16. The method of claim 13, wherein the nitroxide comprises tempol [4-hydroxy-2,2,6,6-tetramethyl-piperidinyloxy, free radical].

17. A topical preparation, oral medication or supplement preparation with enhanced stability or increased activity or longevity produced in accordance with the method of claim 13.

18. A method for reducing or inhibiting growth of microorganisms in topical preparations, said method comprising adding at least one molecule chosen from nitroxides and nitrones to the topical preparation.

19. The method of claim 18, where in the topical preparation is a lotion, gel, solution, cream, ointment, emulsion, or spray.

20. The method of claim 18, wherein the nitroxide comprises tempol [4-hydroxy-2,2,6,6-tetramethyl-piperidinyloxy, free radical].

21. The method of claim 18, wherein the microorganisms are viruses, prions, bacteria, fungi, and yeast.

22. A topical preparation with reduced growth of microorganism produced in accordance with the method of claim 18.
